(19) United States
(12) Patent Application Publication
(10) Pub. No.: US 2024/0079129 A1
(43) Pub. Date: Mar. 7, 2024

(54) COMPREHENSIVE PATIENT BIOLOGICAL INFORMATION COLLECTION DEVICES AND TELEMEDICINE SYSTEMS USING THE SAME

(71) Applicant: XIAO JUN WEN, NORCROSS, GA (US)

(72) Inventor: XIAO JUN WEN, NORCROSS, GA (US)

(21) Appl. No.: 17/901,398

(22) Filed: Sep. 1, 2022

(51) Int. Cl. G16H 40/67; G16H 10/60; G16H 50/20; G16H 80/00

(52) U.S. Cl. CPC G16H 40/67; G16H 10/60; G16H 50/20; G16H 80/00

(57) ABSTRACT

The present disclosure relates to a comprehensive patient biological information collection device (CPBICD). Each patient carries one CPBICD. The CPBICD uses a set of patient biological information collection sensors to collect and monitor patient's biological information constantly. In certain embodiments, CPBICD is used to monitor patients' wellbeing in a telemedicine system. Biological information of each patient is monitored, collected in real time and transmitted to a telemedicine system server for processing using artificial intelligence algorithms to detect early signs of illness. When at least one of the patient biological information collection sensors detects certain type of biological information that exceeds a normal range of baseline biological information stored in a patient database, a telemedicine system determines whether patient needs medical attention, and arranges immediate or future telemedicine sessions according to the availability of healthcare professionals, and provides immediate telemedicine session when needed.

### FIELD

The present disclosure generally relates to medical devices, and more particularly to comprehensive patient biological information collection devices, telemedicine systems having the comprehensive patient biological information collection devices, and the methods of using the telemedicine systems.

### BACKGROUND

In recent years, telehealth and telemedicine market grows rapidly. The global telehealth and telemedicine market is expected to grow at a compound annual growth rate of 26.6% during the forecast period, to reach USD 285.7 billion by 2027 from an estimated USD 87.8 billion in 2022. Telehealth and telemedicine market includes serval segments, software and services, hardware, delivery, application, and end users. Software and service segment has been at the highest rate of growth among all telehealth and telemedicine segments. The pandemic of the current COVID19 further underlines the importance of telehealth and telemedicine and accelerates its growth of all segments.

Despite the rapid growth of telehealth and telemedicine, there are still several challenges that significantly limit its growth and application:

First, lack of user-friendly medical devices to collect critical medical or biological information needed for medical doctors to provide same quality of healthcare or service as the clinic visit. Most patients do not have medical devices that can be easily used to remotely collect and transmit critical medical or biological information to a healthcare provider online or on cloud. Therefore, the current telemedicine are mostly online consultations only and it is a challenge for a clinician to provide necessary healthcare as done in an office visit for majority of the telemedicine due to lack of user-friendly remote medical devices, such as wireless blood pressure meters, ECG, and stethoscopes.

Second, although many smart medical devices have been made for home use and widely available on the market, using such smart medical devices is often a job of trained medical or health professional. Most patients, especially the elderlies, may not know how to operate several medical devices needed at home in order to provide necessary information to the medical doctor online or on cloud.

Third, multiple separated home medical devices make the data collections and transmission complicated and often make it difficult for patients to complete such task to provide critical data to the healthcare providers online or on cloud.

Fourth, home medical devices made by different manufacturers cannot communicate with each other even though they can automatically collect and transmit data to their Apps or clouds, which makes it a challenge for the telemedicine doctors to access the data collected through different devices and platforms. This challenge is critical especially in emergency when full access to vital signs or information in time is needed to save lives.

In addition to the challenges described above, each smart medical device has its one power supply, display screen, computer chips, hardware and software of data collection, procession, and transmission, which is not only waste of resources or environment-unfriendly, but also not cost-effective to the users.

Therefore, a smarter and integrated home medical device for the same critical medical information collections as regularly done in office or clinic visit is urgently needed to overcome the challenges facing the rapid emerging market of telemedicine, especially during the pandemic when office or clinic visit is limited, at the situations where medical care can only be accessed remotely or during the emergency when rescue time is so critical.

Therefore, a heretofore unaddressed needs still exist in the art to address the aforementioned deficiencies and inadequacies.

### SUMMARY

In one aspect, the present disclosure relates to a comprehensive patient biological information collection device. In certain embodiments, the comprehensive patient biological information collection device includes: a processor, a network interface controller, and a non-volatile memory. The processor controls operations of the comprehensive patient biological information collection device. The network interface controller facilitates the communication among the comprehensive patient biological information collection device, a telemedicine system server and a communication system over a communication network.

In certain embodiments, the non-volatile memory stores an operating system, a GPS module for detecting the GPS location of a patient carrying the comprehensive patient biological information collection device, and a comprehensive patient biological information collection device controller having a patient communication module, a comprehensive patient biological information collection module, a comprehensive patient biological information processing module, a comprehensive patient biological information storage module; and computer executable instructions. When executed by the processor, the computer executable instructions perform one or more of following operations:
receiving, through the comprehensive patient biological information collection module over the patient communication module, patient biological information constantly monitored and collected in real time by a set of patient biological information collection sensors of the comprehensive patient biological information collection device;
processing, by the comprehensive patient biological information processing module, the patient biological information received from the set of patient biological information collection sensors;
storing, by the comprehensive patient biological information storage module, the processed patient biological information; and
updating, through the patient communication module, a patient database.

In certain embodiments, the patient communication module facilitates communication among the patient database, the comprehensive patient biological information collection device controller, and the set of patient biological information collection sensors through the network interface controller over the communication network. The comprehensive patient biological information collection module collects patient biological information through a wireless data receiver over the communication network from the set of patient biological information collection sensors from the patient. The comprehensive patient biological information processing module processes the patient biological information collected through the comprehensive patient biological information collection module. The comprehensive patient biological information storage module stores the patient biological information processed through the comprehensive patient biological information processing module and synchronizes the patient biological information stored with the patient database.

In certain embodiments, the communication network includes a wireless personal area network (WPAN) and the wireless personal area network includes a Wi-Fi network, a Bluetooth network, an infrared network, and a Zigbee network, a wireless local area network (WLAN), a wireless metropolitan area network (WMAN), a wireless wide area network (WWAN), a cellular network, and a mobile communication network.

In certain embodiments, the set of patient biological information collection sensors includes one or more of:
a chip reader for reading a patient information card where patient information is stored;
a geolocation sensor for tracking the geolocation of the patient;
a speaker for the patient to make and receive voice calls, and to provide voice instructions to the patient;
a stethoscope sensor for monitoring patient's heart, lung and breathing conditions; a blood oxygen sensor for monitoring patient's blood oxygen level;
a motion sensor for monitoring patient's movement;
an acoustic sensor for the patient to make and receive voice calls, to generate voice samples of the patient, and to detect background sounds surrounding the patient;
a temperature sensor for monitoring patient's temperature;
a blood pressure sensor for monitoring patient's blood pressure;
a blood sugar sensor for monitoring patient's blood sugar level;
an EKG/ECG sensor for monitoring patient's electrocardiograms;
an EEG sensor for monitoring patient's electroencephalograms;
a video camera for monitoring patient's posture and movement;
an accelerometer for detecting impact to the patient and sudden fall of the patient;
an abnormal odor sensor for detecting harmful gas around the patient;
a breathalyzer sensor for detecting blood alcohol level of the patient, and any combinations of such biological information collection sensors.

In another aspect, the present disclosure relates to a telemedicine system. In certain embodiments, the telemedicine system includes: a telemedicine system server, a communication system, a patient database, and a set of comprehensive patient biological information collection devices.

Each comprehensive patient biological information collection device is logged into his/her own registered account and used by one patient at a time. The comprehensive patient biological information collection device collects patients' biological information through a set of patient biological information collection sensors. The set of patient biological information collection sensors constantly monitors the biological information of the patient and transmits the patient biological information collected to the telemedicine system server through the comprehensive patient biological information collection device. When at least one of the patient biological information collection sensors detects at least one abnormal condition when at least one type of biological information exceeds a normal range of the set of baseline biological information stored in the patient database, the telemedicine system server initiates at least voice communication between the telemedicine system server and the comprehensive patient biological information collection device of the patient through the communication system directly to consult the patient.

The patient database is connected to and accessible by the telemedicine system server. The patient database stores patient information of a set of patients. The patient information includes personal information, medical history, a set of baseline biological information, patient contact information, and contact information of relatives and friends on record of each of the set of the patients.

The communication system is connected to the telemedicine system server, and provides voice, text, and video communication over a communication network among the patients, one or more live telemedicine clinicians from a telemedicine center, and one or more patient's relatives and friends on record.

In certain embodiments, a patient condition indicator of the comprehensive patient biological information collection device is lit in green light indicating the comprehensive patient biological information collection device is in normal operation state, and the patient is in normal condition. When at least one abnormal condition is detected from the patient, the patient condition indicator turns red indicating that the patient should consult at least certain healthcare professionals. At this time, the comprehensive patient biological information collection device of the patient initiates a call to the telemedicine center to notify a live telemedicine clinician with the patient's GPS location information and patient information including contact information of patient's relatives and friends on record, the live telemedicine clinician connects to the patient based on the patient information received and retrieved from the patient database. The live telemedicine clinician uses the patient biological information collected to perform a remote diagnose and to use an artificial intelligence algorithm to decide whether an immediate or a future telemedicine session with healthcare professional is necessary using the patient information received and retrieved from the patient database, the patient biological information collected.

In certain embodiments, the telemedicine system server includes: a server processor, a network interface controller, and a non-volatile memory. The server processor controls operations of the telemedicine system. The network interface controller is connected to the communication network through a firewall connector over a firewall. The non-volatile memory stores a server operating system, a network communication module, and a telemedicine system controller. The telemedicine system controller includes a patient information storage module for accessing the patient database through a database interface, a communication control module for facilitating communication to the communication system through a communication control interface, a patient information processing module, and computer executable instructions. When executed by the server processor, the computer executable instructions perform one or more of following operations:
receiving, through a patient communication module of the comprehensive patient biological information collection device of the patient, patient biological information constantly monitored and collected in real time by the set of patient biological information collection sensors of the comprehensive patient biological information collection device;
processing, by the patient information processing module, the patient biological information received;
comparing, by the patient information processing module, the patient biological information processed against base line patient biological information from the patient database using an artificial intelligence algorithm;
initiating, by the telemedicine system server, a voice call to the comprehensive patient biological information collection device of the patient through the communication system, when at least one of the patient biological information collection sensors detects at least one abnormal condition;
initiating, by the telemedicine system server, a call to the telemedicine center to notify the live telemedicine clinician with the patient's GPS location information and patient information including contact information of patient's relatives and friends on record;
contacting, through the communication control module, the patient via the comprehensive patient biological information collection device carried by the patient to perform a remote diagnose and to use an artificial intelligence algorithm to decide whether an immediate or a future telemedicine session with healthcare professional is necessary using the patient information received and retrieved from the patient database, the patient biological information collected;
starting, via the comprehensive patient biological information collection device, the immediate telemedicine session with the patient by the healthcare professionals, if the immediate telemedicine session is necessary; and
setting up, via the comprehensive patient biological information collection device, the future telemedicine session if the immediate telemedicine session is not necessary or required healthcare professionals for the patient is not immediately available.

In certain embodiments, the comprehensive patient biological information collection device also includes: a telemedicine contact button, the patient condition indicator, a display screen, a power supply module, and a SIM card slot. When an immediate telemedicine session is requested by the patient, the patient presses the telemedicine contact button to initiate a contact with the live telemedicine clinician. The patient condition indicator is lit in green indicating that the patient is in normal condition. When at least one abnormal condition is detected on the patient and the patient may need healthcare professional's attention, the patient condition indicator turns in red. The display screen is used for receiving and displaying text messages, emails, diagnostic messages, and making video calls. The power supply module provides electrical power to the comprehensive patient biological information collection device. A SIM card for mobile communication is inserted in the SIM card slot to enable wireless communication.

In certain embodiments, the communication among the patient, the live telemedicine clinician, the patient's relatives and friends on record and nearby medical facilities includes: mobile voice calls, mobile videotelephony calls, landline voice calls, videotelephony calls over the Internet, text messages over a mobile phone, text messages over a set of social media platforms, and videotelephony calls over the set of social media platforms.

In certain embodiments, the communication system includes:
a voice input module having a mobile voice input interface to receive mobile voice calls, and a landline voice input interface to receive landline voice calls;
a text input module having a social media text input interface to receive text messages through the set of social media platforms, and a text message input interface to receive text messages through mobile phones;
a video input module having a mobile video input interface to receive video calls over the mobile phones, and a social media video input interface to receive video calls through the set of social media platforms;
a voice output module having a mobile voice output interface to make mobile voice calls, and a landline voice output interface to make landline voice calls;
a text output module having a social media text output interface to transmit text messages through the set of social media platforms, and a text message output interface to transmit text messages through the mobile phones;
a video output module having a mobile video output interface to make video calls over the mobile phones, and a social media video output interface to make video calls through the set of social media platforms;
a speech to text conversion module for converting voice input to text input;
a live telemedicine clinician interface module for the live telemedicine clinician to receive and make conference calls among the patient, the live telemedicine clinician, the patient's relatives and friends on record and the nearby medical facility through voice calls, text messages, and video calls; and
a text to speech conversion module to make voice calls to the patient through the comprehensive patient biological information collection device.

In certain embodiments, the communication control interface includes:
a voice input terminal connected to the voice input module;
a text input terminal connected to the text input module;
a video input terminal connected to the video input module;
a voice output terminal connected to the voice output module;
a text output terminal connected to the text output module; and
a video output terminal connected to the video output module.

In certain embodiments, the comprehensive patient biological information collection device includes:
a portable comprehensive patient biological information collection device to be carried with the patient;
a stationary comprehensive patient biological information collection device to be placed at home or work place of the patient and to be shared with one or more registered patients; and
a set of public stationary comprehensive patient biological information collection devices to be placed in public places and to be shared with one or more registered patients.

In yet another aspect, the present disclosure relates to a method of using a telemedicine system. In certain embodiments, the method includes
registering, by a set of patients, the set of patients, and a set of comprehensive patient biological information collection devices at a telemedicine system server of the telemedicine system, wherein patient information of each of the set of patients is stored in a patient database of the telemedicine system, and the telemedicine system provides telemedicine assistance to the patient by a healthcare professional when a comprehensive patient biological information collection device of the patient detects at least one abnormal condition when at least one type of biological information exceeds a normal range of the set of baseline biological information stored in the patient database;
constantly monitoring and collecting in real time, by a set of patient biological information collection sensors of the set of the comprehensive patient biological information collection devices, patient biological information of each patient, where the patient logs into his/her own registered account;
receiving, through a patient communication module from the comprehensive patient biological information collection device, the patient biological information constantly monitored and collected in real time by the set of patient biological information collection sensors of the comprehensive patient biological information collection device;
processing, through a patient information processing module of the telemedicine system server of the telemedicine system, the patient biological information received, and comparing the patient biological information received with a set of baseline biological information stored in the patient database using an artificial intelligence algorithm;
initiating, by the telemedicine system server, a voice call to the comprehensive patient biological information collection device of the patient through a communication system, when at least one of the patient biological information collection sensors detects at least one abnormal condition;
initiating, by the telemedicine system server, a call to the telemedicine center to notify the live telemedicine clinician with the patient's GPS location information and patient information including contact information of patient's relatives and friends on record;
contacting, through a communication control module of a telemedicine system controller of the telemedicine system server, the patient via the comprehensive patient biological information collection device carried by the patient to perform a remote diagnose and to use an artificial intelligence algorithm to decide whether an immediate or a future telemedicine session with healthcare professional is necessary using the patient information received and retrieved from the patient database, the patient biological information collected;
starting, via the comprehensive patient biological information collection device, the immediate telemedicine session with the patient by the healthcare professionals, if the immediate telemedicine session is necessary; and
setting up, via the comprehensive patient biological information collection device, the future telemedicine session if the immediate telemedicine session is not necessary or required healthcare professionals for the patient is not immediately available.

In certain embodiments, the starting, via the comprehensive patient biological information collection device, the immediate telemedicine session with the patient by the healthcare professionals includes:
establishing voice communication, text messages or video communication, via the communication system of the telemedicine system, between a healthcare professional and the patient;
connecting, via the communication system of the telemedicine system, the plurality of patient biological information collection sensors through the comprehensive patient biological information collection device; and
performing, by the healthcare professional via the comprehensive patient biological information collection device, a diagnose of the patient using the patient's biological information retrieved from the patient database, the patient's biological information collected through the plurality of patient biological information collection sensors, and the feedback from the patient; and
providing timely treatment, by the healthcare professional via the comprehensive patient biological information collection device, to the patient.

These and other aspects of the present disclosure will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the present disclosure, and features and benefits thereof, and together with the written description, serve to explain the principles of the present invention. Wherever possible, the same reference numbers are used throughout the drawings to refer to the same or like elements of an embodiment, and wherein:

FIG. 1 shows a block diagram of a comprehensive patient biological information collection device according to certain embodiments of the present disclosure;

FIG. 2 shows a block diagram of a patient biological information collection module of the comprehensive patient biological information collection device using a set of patient biological information collection sensors to collect patient's biological information according to certain embodiments of the present disclosure;

FIG. 3 illustrates an exemplary set of patient biological information collection sensors used to collect patient biological information according to certain embodiments of the present disclosure;

FIG. 4 shows an exemplary comprehensive patient biological information collection device according to certain embodiments of the present disclosure;

FIG. 5 shows an exemplary robot as a patient biological information collection sensor for collecting patient biological information according to certain embodiments of the present disclosure;

FIG. 6 illustrates a block diagram of a telemedicine system using a set of comprehensive patient biological information collection devices (CPBICD) according to certain embodiments of the present disclosure;

FIG. 7 illustrates a block diagram of a telemedicine system server of the telemedicine system according to certain embodiments of the present disclosure;

FIG. 8 illustrates a block diagram of a communication system of the telemedicine system according to certain embodiments of the present disclosure; and

FIG. 9 shows a flow chart of a method of using the telemedicine system using the set of patient biological information collection sensors according to certain embodiments of the present disclosure.

### DETAILED DESCRIPTION

The present disclosure is more particularly described in the following examples that are intended as illustrative only since numerous modifications and variations therein will be apparent to those skilled in the art. Various embodiments of the disclosure are now described in detail. Referring to the drawings, like numbers, if any, indicate like components throughout the views. As used in the description herein and throughout the claims that follow, the meaning of “a”, “an”, and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which shall have no influence on the scope of the present disclosure. Additionally, some terms used in this specification are more specifically defined below.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the disclosure or of any exemplified term. Likewise, the disclosure is not limited to various embodiments given in this specification.

Unless otherwise defined, all technical and scientific terms used herein have the same meaning as commonly understood by one of ordinary skill in the art to which this disclosure pertains. In the case of conflict, the present document, including definitions will control.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

As used herein, “plurality” means two or more.

As used herein, the terms “comprising,” “including,” “carrying,” “having,” “containing,” “involving,” and the like are to be understood to be open-ended, i.e., to mean including but not limited to.

As used herein, the phrase at least one of A, B, and C should be construed to mean a logical (A or B or C), using a non-exclusive logical OR. It should be understood that one or more steps within a method may be executed in different order (or concurrently) without altering the principles of the present disclosure.

As used herein, the term module may refer to, be part of, or include an Application Specific Integrated Circuit (ASIC); an electronic circuit; a combinational logic circuit; a field programmable gate array (FPGA); a processor (shared, dedicated, or group) that executes code; other suitable hardware components that provide the described functionality; or a combination of some or all of the above, such as in a system-on-chip. The term module may include memory (shared, dedicated, or group) that stores code executed by the processor.

The term code, as used above, may include software, firmware, and/or microcode, and may refer to programs, routines, functions, classes, and/or objects. The term shared, as used above, means that some or all code from multiple modules may be executed using a single (shared) processor. In addition, some or all code from multiple modules may be stored by a single (shared) memory. The term group, as used above, means that some or all code from a single module may be executed using a group of processors. In addition, some or all code from a single module may be stored using a group of memories.

The apparatuses and methods described herein may be implemented by one or more computer programs executed by one or more processors. The computer programs include processor-executable instructions that are stored on a non-transitory tangible computer readable medium. The computer programs may also include stored data. Non-limiting examples of the non-transitory tangible computer readable medium are nonvolatile memory, magnetic storage, and optical storage.

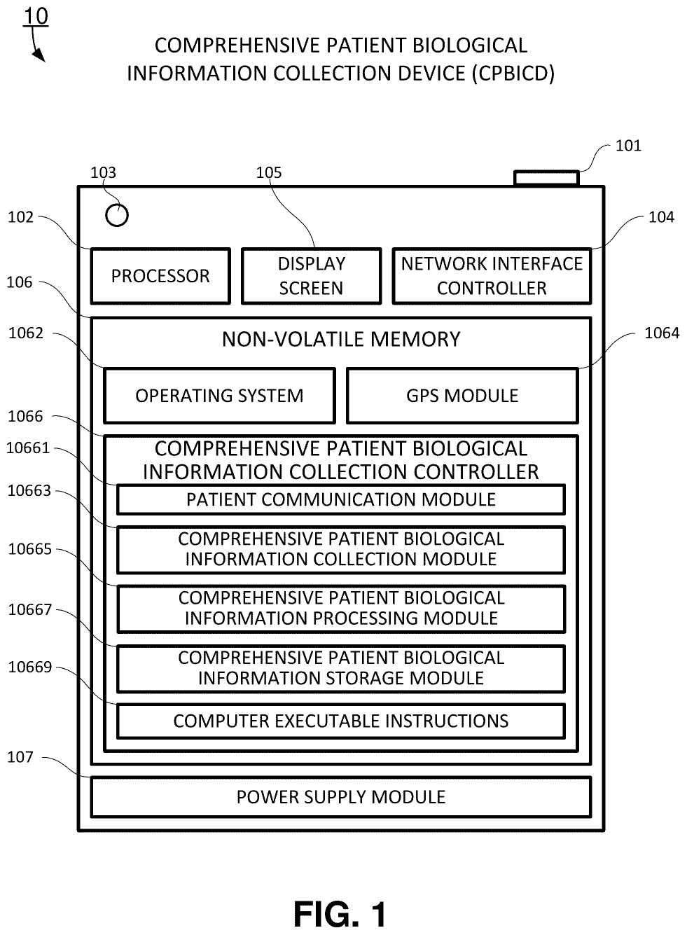

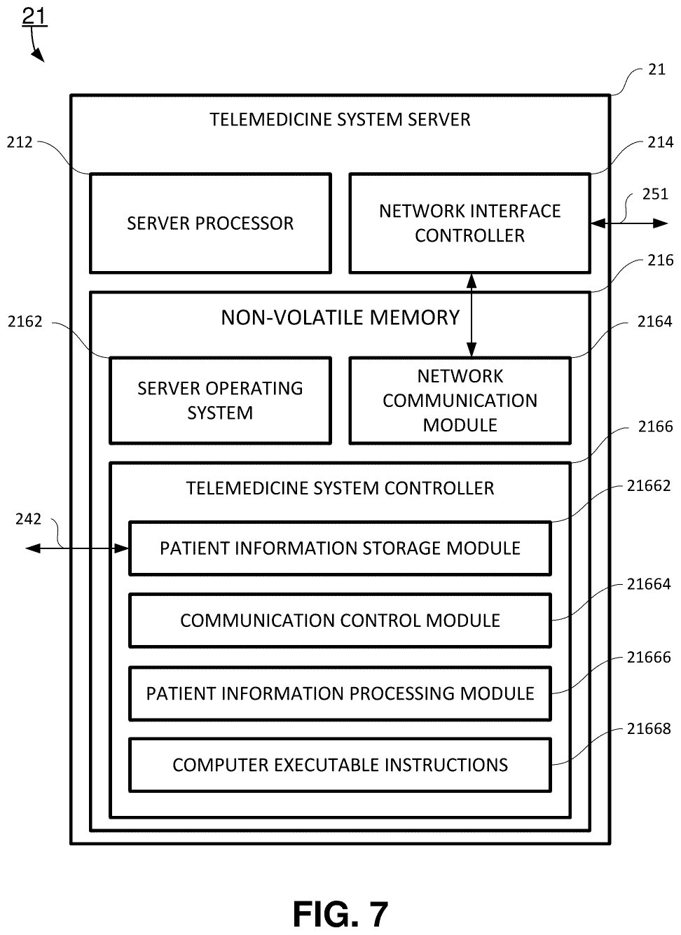

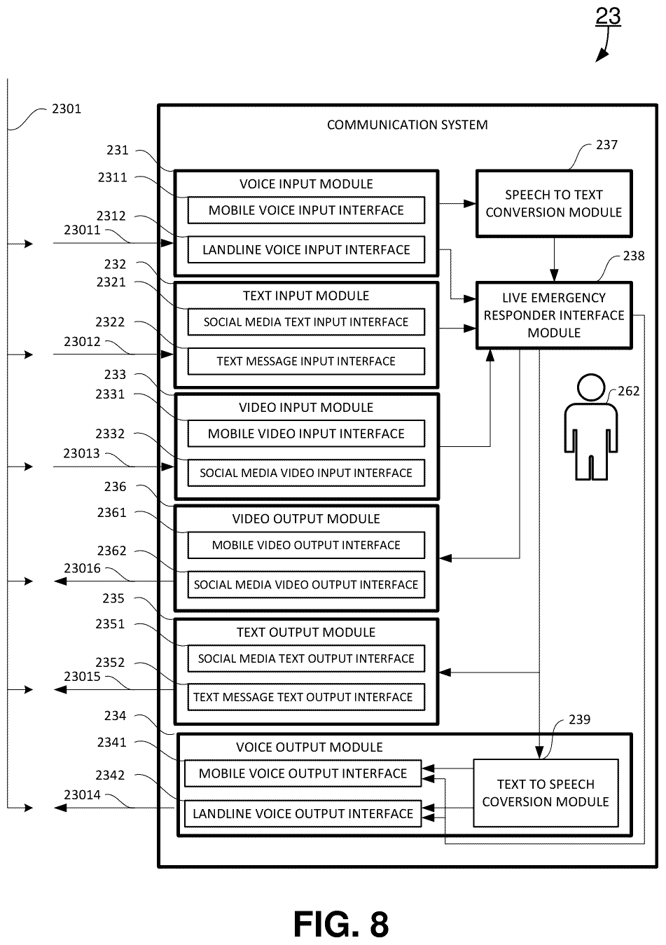

The present disclosure will now be described more fully hereinafter with reference to the accompanying drawings FIGS. 1 through 9, in which embodiments of the disclosure are shown. This disclosure may, however, be embodied in many different forms and should not be construed as limited to the embodiments set forth herein; rather, these embodiments are provided so that this disclosure will be thorough and complete, and will fully convey the scope of the disclosure to those skilled in the art. Like numbers refer to like elements throughout.

Currently, there is no comprehensive patient biological information collection device in the market. In order to collect patient biological information such as: blood pressure, blood sugar, blood alcohol, temperature, EEG, and EKG, many different machines/devices such as blood pressure monitors, blood sugar monitors, breathalyzers, thermometers, EEG monitors, and EKG monitors are needed. The costs of these machines/devices are very high and will not meet the low-cost requirement for normal daily monitoring for general population. It is desirable to have a low cost alternative to monitor these patient biological information without the prohibitive costs of a collection of very expensive machines/devices.

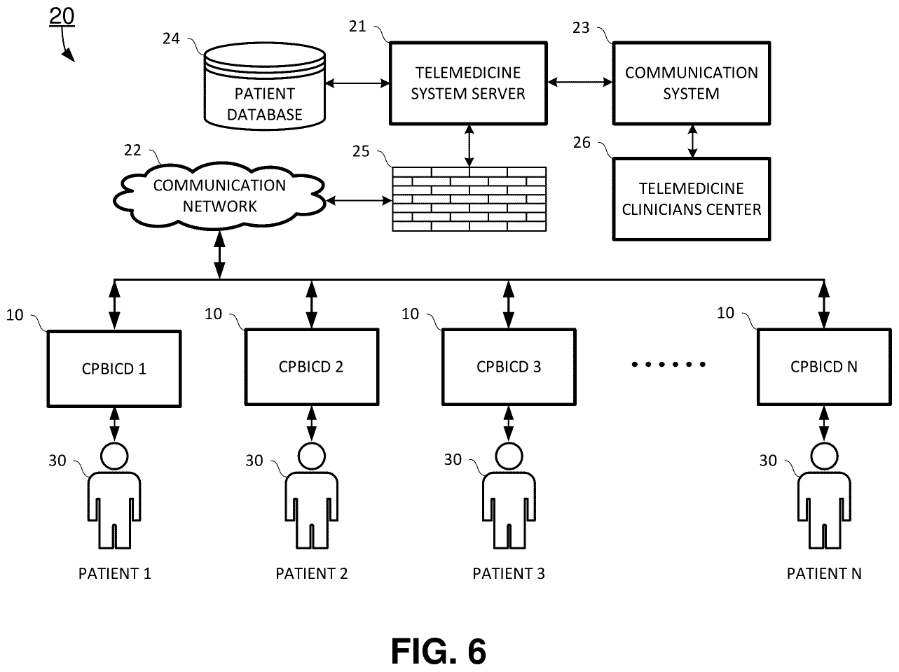

In one aspect, the present disclosure relates to a comprehensive patient biological information collection device 10, as shown in FIGS. 1-5. A group of the comprehensive patient biological information collection devices 10 is used by a group of patients 30. Each comprehensive patient biological information collection device 10 may be shared by one or more patients 30. For Example, one family may have more than one family members and this family does not need to purchase more than one comprehensive patient biological information collection device 10. As long as the comprehensive patient biological information collection device 10 is registered at a telemedicine system 20, as shown in FIG. 6, and each family member registers one account at the telemedicine system 20, one comprehensive patient biological information collection device 10 will be sufficient for the entire family.

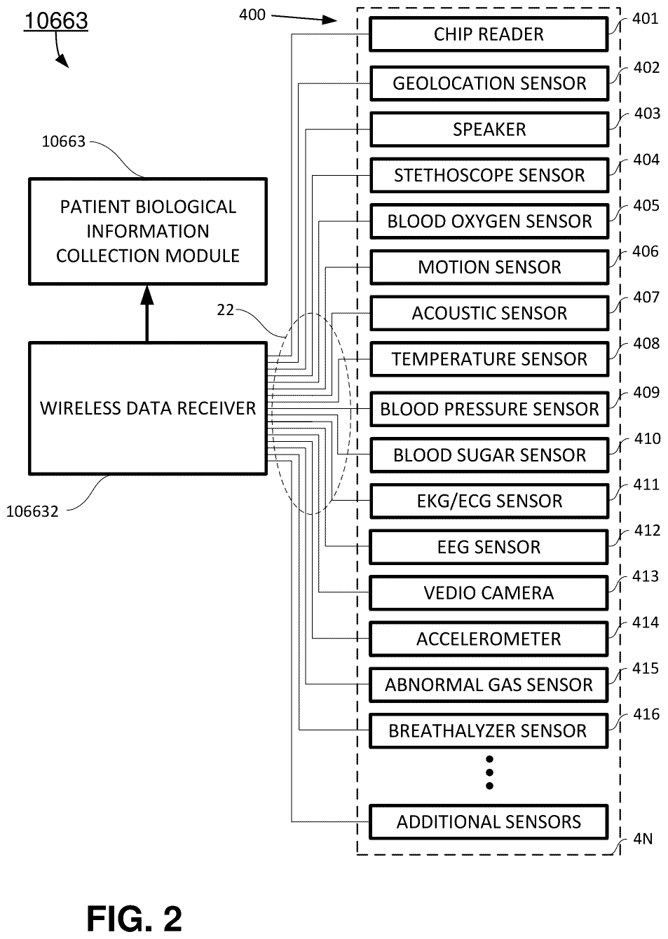

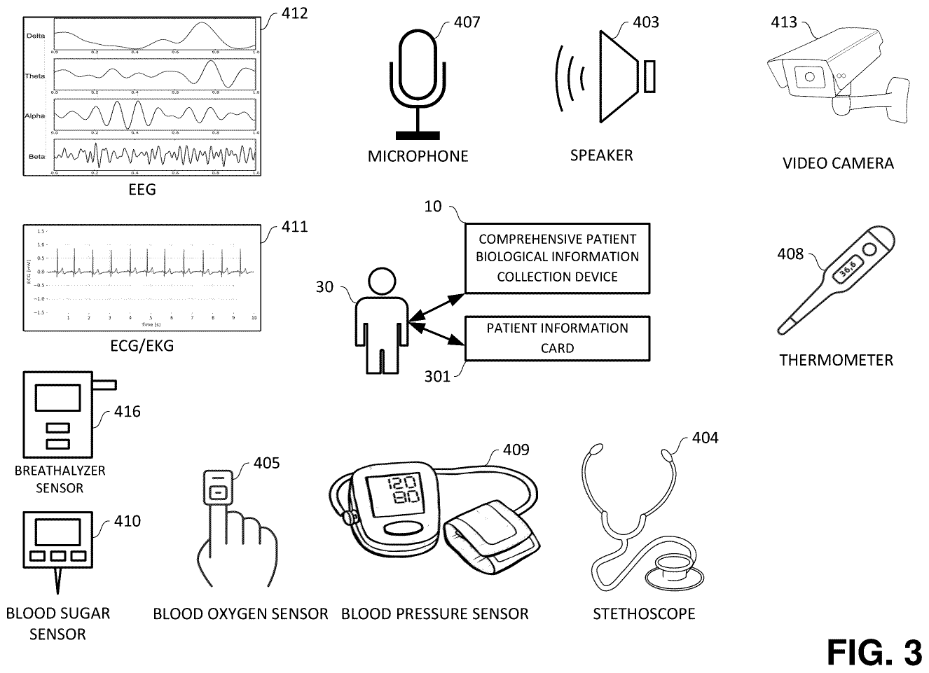

In certain embodiments, the comprehensive patient biological information collection device 10, as shown in FIG. 1, collects biological information of a patient 30 through a set of patient biological information collection sensors 400, as shown in FIG. 2 and FIG. 3, and monitors the wellbeing of the patient 30. The comprehensive patient biological information collection device 10 includes: a processor 102, a network interface controller 104, and a non-volatile memory 106. The processor 102 controls operations of the comprehensive patient biological information collection device 10. The network interface controller 104 facilitates the communication among the comprehensive patient biological information collection device 10, the set of patient biological information collection sensors 400, a telemedicine system server 21 of the telemedicine system 20 and a communication system 23 over a communication network 22.

In certain embodiments, the non-volatile memory 106 stores an operating system 1062, a GPS module 1064 for detecting the GPS location of the patient 30 carrying the comprehensive patient biological information collection device 10, and a comprehensive patient biological information collection device controller 1066 having a patient communication module 10661, a comprehensive patient biological information collection module 10663, a comprehensive patient biological information processing module 10665, a comprehensive patient biological information storage module 10667; and computer executable instructions 10669. The set of patient biological information collection sensors 400 collects patient's biological information through the comprehensive patient biological information collection module 10663, and processes the patient's biological information through the comprehensive patient biological information processing module 10665, in such a way that costs of monitoring patient's biological information is dramatically reduced because all processing of the data collected by the set of patient biological information collection sensors 400 is collectively processed through the processor 102 and the comprehensive patient biological information processing module 10665, instead of processed by each and every individual biological information monitoring machines/devices.

In certain embodiments, when executed by the processor 102, the computer executable instructions 10669 perform one or more of following operations:
receiving, through the comprehensive patient biological information collection module 10663 over the patient communication module 10661, patient biological information constantly monitored and collected in real time by the set of patient biological information collection sensors 400 of the comprehensive patient biological information collection device 10;
processing, by the comprehensive patient biological information processing module 10665, the patient biological information received from the set of patient biological information collection sensors 400;
storing, by the comprehensive patient biological information storage module 10667, the processed patient biological information; and
updating, through the patient communication module 10661, a patient database 24 of the telemedicine system 20.

In certain embodiments, the patient communication module 10661 facilitates communication among the patient database 24, the comprehensive patient biological information collection device controller 1066, and the set of patient biological information collection sensors 400 through the network interface controller 104 over the communication network 22. As shown in FIG. 2, the comprehensive patient biological information collection module 10663 collects patient biological information through a wireless data receiver 106632 over the communication network 22 from the set of patient biological information collection sensors 400 from the patient 30. The comprehensive patient biological information processing module 10665 processes the patient biological information collected through the comprehensive patient biological information collection module 10663. The comprehensive patient biological information storage module 10667 stores the patient biological information processed through the comprehensive patient biological information processing module 10665 and synchronizes the patient biological information stored with the patient database 24.

In certain embodiments, the communication network 22 includes a wireless personal area network (WPAN). The wireless personal area network includes a Wi-Fi network, a Bluetooth network, an infrared network, a Zigbee network, a wireless local area network (WLAN), a wireless metropolitan area network (WMAN), a wireless wide area network (WWAN), a cellular network, and a mobile communication network.

In certain embodiments, as shown in FIG. 2 and FIG. 3, the set of patient biological information collection sensors 400 includes one or more of: a chip reader 401, a geolocation sensor 402, a speaker 403, a stethoscope sensor 404, a blood oxygen sensor 405, a motion sensor 406, an acoustic sensor 407, a temperature sensor 408, a blood pressure sensor 409, a blood sugar sensor 410, an EKG/ECG sensor 411, an EEG sensor 412, a video camera 413, an accelerometer 414, an abnormal odor sensor 415, and a breathalyzer sensor 416.

In certain embodiments, patient information can be stored in a credit card sized patient information card 301, as shown in FIG. 3. The patient information includes personal information, medical history, a set of baseline biological information, patient contact information, and contact information of relatives, friends and other responders and local medical facilities to be notified of each registered patient 30. When registering the comprehensive patient biological information collection device 10 and the personal account by the patient 30, the chip reader 401 can be used to upload important patient information into the comprehensive patient biological information collection device 10 and the patient database 24 of the telemedicine system 20 very quickly without typing errors.

In certain embodiments, the geolocation sensor 402 is used to monitor the geolocation of the patient 30. Such geolocation sensor 402 can be a part of the comprehensive patient biological information collection device 10, an electronic watch, or a wearable device wirelessly connected to the comprehensive patient biological information collection device 10. It tracks the movement of the patient 30, and enables the telemedicine system 20 to locate the patient 30 at any time. If the patient 30 gets into certain predetermined areas, it may trigger an undesirable condition for the patient 30 that certain people in his/her contact list may be notified.

In certain embodiments, the speaker 403, as well as an earphone, is used for the patient 30 to make and receive voice calls, and to provide voice instructions to the patient 30 for the purposes of diagnose and treatments.

In certain embodiments, the stethoscope sensor 404 may be a separate device and will be used only when requested by a medical professional during a remote diagnose or medical treatment. When a remote medical professional or a live telemedicine clinician 262 needs to check and monitor patient's heart, lung and breathing conditions, the patient 30 may be instructed to place the stethoscope sensor 404 in designated area of interest for the examination and monitoring.

In certain embodiments, as shown in FIG. 3, the stethoscope sensor 414 is used for monitoring breathing, heart, murmurs, Atrial Fibrillation (AFib), thoracic, arterial, intravenous, uterine, fetal, intestinal and other sounds of the patient 30. The stethoscope sensor 414 collects and monitors breathing, heart, thoracic, arterial, intravenous, uterine, fetal, intestinal and other sounds of the patient 30, then various sound samples collected are transmitted to the comprehensive patient biological information collection module 10663 and the comprehensive patient biological information processing module 10665 of the comprehensive patient biological information collection device 10 for sound signal processing using artificial intelligence algorithms. When the various sound samples received and analyzed deviate substantially from the normal baseline corresponding sound samples, the comprehensive patient biological information collection device 10 determines that the patient 30 is near or in an undesirable health condition, and the live telemedicine clinician 262 or medical professionals may be notified.

In certain embodiments, the blood oxygen sensor 405 is used for monitoring patient's blood oxygen level. The blood oxygen sensor 405 may not connected to the patient 30 constantly, and may be used when directed by a medical professional or a live telemedicine clinician 262. In certain embodiments, the blood oxygen sensor 405 may be used on a daily basis to monitor the patient's blood oxygen level.

In certain embodiments, the motion sensor 406 is widely used for monitoring patient's movement. Such the motion sensor 406 can be a part of the comprehensive patient biological information collection device 10, an electronic watch, or a wearable device wirelessly connected to the comprehensive patient biological information collection device 10. When the patient 30 moves in an unusual pace, speed, or sometimes combined with the accelerometer 414 when an impact to the patient 30 is detected, an abnormal condition may be triggered so that certain people in his/her contact list may be notified.

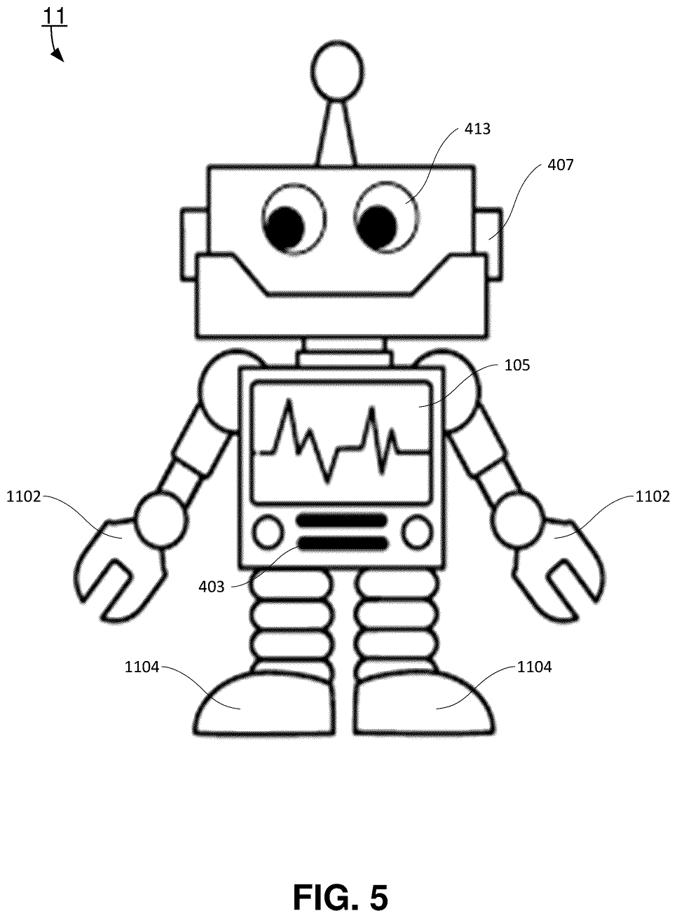

In certain embodiments, the acoustic sensor 407, or a microphone, is used to make and receive voice calls by the patient 30. The acoustic sensor 407 can be installed in the comprehensive patient biological information collection device 10, or a robot 11, as shown in FIG. 5. The acoustic sensor 407 may be used to generate voice samples of the patient 30 for diagnostic purposes. When the patient 30 speaks at an unusual pace, unclear, or sluggish, it may trigger an abnormal condition that certain people in his/her contact list may be notified. In certain embodiments, the acoustic sensor 407 may be used by the live telemedicine clinician 262 or medical professionals to detect background sounds surrounding the patient 30.

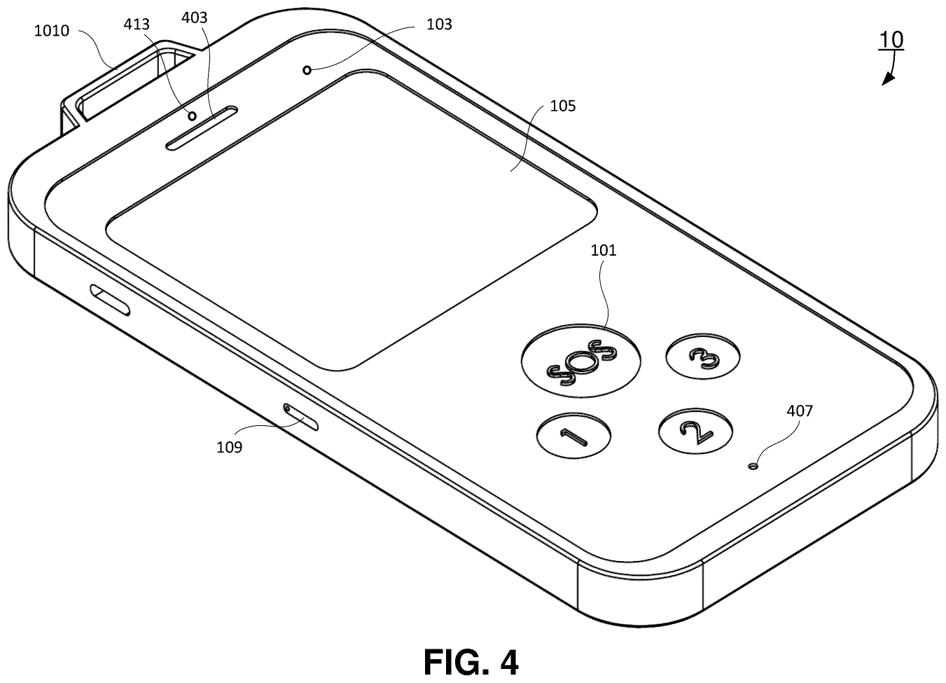

In certain embodiments, as shown in FIGS. 4 and 5, the acoustic sensor 407 (or a microphone) and the speaker 403 are used to allow the patient 30 to make and receive voice calls, to generate voice samples of the patient 30, and to provide voice instructions to the patient 30. The acoustic sensor 407 may also be used to collect speech samples of the patient 30. When the patient 30 is experiencing difficulty speaking, the speech samples collected will deviate from the normal speech patterns, for example, the tempo of the speech, the pitch, the intensity may change dramatically. These deviations may be picked up through the comprehensive patient biological information collection module 10663 and the comprehensive patient biological information processing module 10665 of the comprehensive patient biological information collection device 10 through speech processing using artificial intelligence algorithms. When these speech parameters such as the tempo, the pitch, and the intensity are determined to exceed a normal range of these speech parameters, the comprehensive patient biological information collection device 10 determines that the patient 30 is near or in an abnormal condition, and the live telemedicine clinician 262 or medical professionals may be notified.

In certain embodiments, the voice data by the acoustic sensor 407 from the patient 30 may be used as patient's identifier the same way as the fingerprinting as the fingerprinting to determine the user's identity.

In certain embodiments, the temperature sensor 408 is used for monitoring patient's temperature. Such the temperature sensor 408 can be a part of the comprehensive patient biological information collection device 10, an electronic watch, or a wearable device wirelessly connected to the comprehensive patient biological information collection device 10. One or more temperature sensors may be placed in predetermined locations of the patient 30. These sensors collect and monitor body temperatures of the patient 30 in predetermined intervals. The temperatures collected are transmitted to the comprehensive patient biological information collection module 10663 and the comprehensive patient biological information processing module 10665 of the comprehensive patient biological information collection device 10 for temperature processing using artificial intelligence algorithms. When the temperatures received and analyzed deviate substantially from the normal baseline corresponding temperature data, or body temperature of the patient 30 exhibits sudden change, the comprehensive patient biological information collection device 10 determines that the patient 30 is near or in an abnormal condition and the live telemedicine clinician 262 or medical professionals may be notified.

In certain embodiments, the blood pressure sensor 409 is used for monitoring patient's blood pressure and pulse rate. Such the blood pressure sensor 409 can be a part of the comprehensive patient biological information collection device 10, an electronic watch, or a wearable device wirelessly connected to the comprehensive patient biological information collection device 10. The blood pressure sensor 409 can ensure the heart and blood vessel's health of the patient 30 and detect abnormal condition before a stroke or heart attack happens. When blood pressure of the patient 30 exceeds certain normal range, an abnormal condition may be triggered and the live telemedicine clinician 262 or medical professionals may be notified.

In certain embodiments, the blood sugar sensor 410 is used for monitoring patient's blood sugar level. Such the temperature sensor 408 can be a part of the comprehensive patient biological information collection device 10, an electronic watch, or a wearable device wirelessly connected to the comprehensive patient biological information collection device 10. The blood sugar sensor 410 is very important electronic equipment for diabetic patients. When blood sugar level of the patient 30 exceeds certain normal range, an abnormal condition may be triggered and the live telemedicine clinician 262 or medical professionals may be notified.

In certain embodiments, the EKG/ECG sensor 411 is used for monitoring patient's electrocardiograms. Such EKG/ECG sensor 411 can be a part of the comprehensive patient biological information collection device 10, an electronic watch, or a wearable device wirelessly connected to the comprehensive patient biological information collection device 10. When electrocardiograms patterns of the patient 30 indicate any abnormality, an abnormal condition may be triggered and the live telemedicine clinician 262 or medical professionals may be notified.

In certain embodiments, as shown in FIG. 3, the electrocardiograms 1405 are used for monitoring electrocardiography of the patient 30. Full featured electrocardiograms 1405 may be found from doctor's offices, however, basic electrocardiograms 1405 may be found in wearable mobile devices such as Apple Watches. The patient 30 can use the full-featured electrocardiograms 1405 occasionally in doctor's office, a smart phone-based electrocardiograms 1405 may be used to monitor electrocardiography data in a continuous manner. The Apple Watch includes sensors to collect and monitor the electrocardiography data of the patient 30, then the electrocardiography data collected is wirelessly transmitted to the comprehensive patient biological information collection device 10. The electrocardiography data collected is transmitted and processed at the comprehensive patient biological information collection module 10663 and the comprehensive patient biological information processing module 10665 of the comprehensive patient biological information collection device 10 for electrocardiography data processing using artificial intelligence algorithms. When the electrocardiography data received and analyzed deviates substantially from the normal baseline electrocardiography data, the comprehensive patient biological information collection device 10 determines that the patient 30 is near or in an abnormal condition, and the live telemedicine clinician 262 or medical professionals may be notified.

In certain embodiments, the EEG sensor 412 is used for monitoring patient's electroencephalograms. Such EEG sensor 412 can be a part of the comprehensive patient biological information collection device 10, an electronic watch, or a wearable device wirelessly connected to the comprehensive patient biological information collection device 10. When electroencephalograms patterns of the patient 30 indicate any abnormality, an abnormal condition may be triggered and the live telemedicine clinician 262 or medical professionals may be notified.

In certain embodiments, as shown in FIG. 3, the electroencephalograms are used for monitoring electroencephalography of the patient 30. Due to the expensive nature of the electroencephalograms, the patient 30 may not be able to afford to have an electroencephalogram installed at home or office. However, the comprehensive patient biological information collection device 10 may include wireless interfaces to the electroencephalogram installed in doctor's office, and the patient 30 may be able to go to doctor's office regularly and obtain electroencephalography of the patient 30. The comprehensive patient biological information collection device 10 receives electroencephalography data and transmits the electroencephalography data received to the comprehensive patient biological information collection module 10663 and the comprehensive patient biological information processing module 10665 of the comprehensive patient biological information collection device 10 for electroencephalographical data processing using artificial intelligence algorithms. When the electroencephalographical data received and analyzed deviates substantially from the normal baseline electroencephalographical data, the comprehensive patient biological information collection device 10 determines that the patient 30 is near or in an abnormal condition, and the live telemedicine clinician 262 or medical professionals may be notified.

In certain embodiments, the video camera 413 is used for monitoring patient's posture, walking patterns and movement. The video camera 413 can be installed in a home, on the comprehensive patient biological information collection device 10, as shown in FIG. 4, or on a robot 11, as shown in FIG. 5. The video camera 413 also allows the patient 20 to conduct video conference with caretakers, the live telemedicine clinician 262 or medical professionals. When an unusual walking pattern, or unusual posture is detected by the video camera 413, an abnormal condition may be triggered and the live telemedicine clinician 262 or medical professionals may be notified.

In certain embodiments, the image or video data collected from the patient 30 may be used as for facial recognition to login to the personal account of the patient 30 the same way as the fingerprinting to determine the patient's identity. It is very helpful for switching users when a family shares one comprehensive patient biological information collection device 10.

In certain embodiments, as shown in FIG. 4 and FIG. 5, the video camera 413 may be used with a display screen 105 to receive and display text messages and carry out video calls. Additionally, the video camera 413 are also used to monitoring the patient 30 through videos and still images. In one embodiment, a video camera 413 is equipped on the comprehensive patient biological information collection device 10, as shown in FIG. 4, or on a robot 11. In another embodiments, a video camera 413 is installed on the wall of a room the patient stays in most of times, such as a room in a house, or room in an office. The wall-mount video camera 413 may be wirelessly connected to the comprehensive patient biological information collection device 10. The video camera 413 are used to collect videos and still images of the patient 30 during his/her daily life. The baseline biological information of the patient 30 includes normal facial images and normal walking videos of the patient 30 from many different angles and directions. Pre-stroke patient may experience certain facial expression changes such as facial paralysis, and walking patterns may also change. These deviations may be picked up through the comprehensive patient biological information collection module 10663 and the comprehensive patient biological information processing module 10665 of the comprehensive patient biological information collection device 10 through image and video processing using artificial intelligence algorithms. When these facial expressions and walking patterns of the patient 30 are determined to exceed a normal range of baseline images or baseline videos, the comprehensive patient biological information collection device 10 determines that the patient 30 is near or in an abnormal condition, and the live telemedicine clinician 262 or medical professionals may be notified.

In certain embodiments, the accelerometer 414 is used for detecting impact to the patient 30 and sudden fall of the patient 30. Such accelerometer 414 can be a part of the comprehensive patient biological information collection device 10, an electronic watch, or a wearable device wirelessly connected to the comprehensive patient biological information collection device 10. When the accelerometer 414 detects certain impact to the patient 30, or sudden fall of the patient 30, an abnormal condition may be triggered and the live telemedicine clinician 262 or medical professionals may be notified.

In certain embodiments, the abnormal odor sensor 415 is not necessarily a part of patient's biological information but it is related to the wellbeing of the patient 30. The abnormal odor sensor 415 is used for detecting harmful gas around the patient 30. When harmful gas such as natural gas, gasoline vapor, carbon monoxide, carbon dioxide is detected in higher than normal concentration around the patient 30, an abnormal condition may be triggered and the live telemedicine clinician 262 or medical professionals may be notified.

In certain embodiments, the breathalyzer sensor 416 is used for detecting blood alcohol level of the patient 30. The breathalyzer sensor 416 can be installed in the comprehensive patient biological information collection device 10 carried by the patient 30, or in a car driven by the patient 30, or a wearable device wirelessly connected to the comprehensive patient biological information collection device 10. When the patient 30 is detected to have unusual high blood alcohol level, the comprehensive patient biological information collection device 10 may disable the car driven by the patient 30 wirelessly, and an abnormal condition may be triggered and the live telemedicine clinician 262 or medical professionals may be notified.

In certain embodiments, as shown in FIG. 3, the breathalyzer sensor 416 is used to analyze breath samples of the patient 30. The breathalyzer sensor 416 tests many elements such as blood alcohol level, nicotine, hard to digest food components such as lactose, fructose, sorbitol, and inulin to monitor the overall health of the patient 30. The breathalyzer sensor 416 can be made in a small portable package and wirelessly connected to the comprehensive patient biological information collection device 10 through home network, Wi-Fi, and Bluetooth. The results of analyzed breath samples are transmitted to the comprehensive patient biological information collection module 10663 and the comprehensive patient biological information processing module 10665 of the comprehensive patient biological information collection device 10 for breath sample data processing using artificial intelligence algorithms. When the analyzed breath sample data received and analyzed deviates substantially from the normal baseline breath sample data, the comprehensive patient biological information collection device 10 determines that the patient 30 is near or in an abnormal condition, and the live telemedicine clinician 262 or medical professionals may be notified.

In one embodiment, as shown in FIG. 5, the comprehensive patient biological information collection device 10 is a robot 11. The robot 11 has at least one pair of robotic arms 1102 to perform certain medical assistance such as carrying out certain medical tests. The robot 11 also has at least one pair of robotic feet 1104 to follow the patient 30 around his/her home and/or office, and monitor the patient 30's biological information. The robot 11 has eyes (video cameras 413), ears (microphone 407) and mouth (speakers 403) to monitor the patient facial expression, walking, and speaking. Following patient behaviors may be considered abnormal, and it is worth further investigation: (1) when the facial expressions become very animated and unsymmetrically, (2) when the patient drags his feet, walking much slower than usual, or having difficulty walking, (3) when the patient having difficulty speaking clearly, talking much slower than usual, having difficulty choosing the words, and having difficulty carrying out normal conversation. Under these circumstances, the robot 11 may offer some assistance to measure the blood pressure, heart beat rate, blood sugar level, blood oxygen level using various sensors over the robot 11. After preliminary examinations, and when the robot 11 determines that the patient 30 is in danger of suffering severer medical issues, the robot 11 may contact the live telemedicine clinician 262 or medical professionals for assistance.

In another embodiment, the comprehensive patient biological information collection device 10 is a portable device. The portable comprehensive patient biological information collection device 10 is carried with the patient 30. In yet another embodiment, the comprehensive patient biological information collection device 10 is a stationary comprehensive patient biological information collection device. The stationary comprehensive patient biological information collection device is placed at home or work place and shared by family members or colleagues. In yet another embodiment, the comprehensive patient biological information collection device 10 can be a group of public stationary comprehensive patient biological information collection device. These public stationary comprehensive patient biological information collection devices are placed in public places and shared by any registered patients 30.

In another aspect, the present disclosure relates to a telemedicine system 20, as shown in FIG. 6-FIG. 9. In one embodiment, the telemedicine system 20 can be triggered by a patient pressing a telemedicine contact button 101 when the patient 30 realizes that he/she may be in need of certain medical attention by a healthcare professional, and the patient 30 is still conscious and able to press the telemedicine contact button 101. In another embodiment, when the patient 30 falls unconscious, and unable to press the telemedicine contact button 101, a comprehensive patient biological information collection device 10 carries by the patient 30 may automatically trigger the telemedicine system 20. The comprehensive patient biological information collection device 10 constantly monitors certain patient biological information, and triggers telemedicine system 20 when the comprehensive patient biological information collection device 10 detects certain type of abnormal condition when at least one type of biological information exceeds a normal range of the set of baseline biological information stored in the patient database 24.

In certain embodiments, as shown in FIG. 6, the telemedicine system 20 includes: a telemedicine system server 21, a communication system 23, a patient database 24, and a set of comprehensive patient biological information collection devices 10.

In certain embodiments, the set of the comprehensive patient biological information collection devices 10 is used by a group of patients 30. Each comprehensive patient biological information collection device 10 may be shared by one or more patients 30. Each comprehensive patient biological information collection device 10 is logged into his/her own registered account and used by one patient 30 at a time. For Example, one family may have more than one family members and this family does not need to purchase more than one comprehensive patient biological information collection device 10. As long as the comprehensive patient biological information collection device 10 is registered at a telemedicine system 20, as shown in FIG. 6, and each family member registers one account at the telemedicine system 20, one comprehensive patient biological information collection device 10 will be sufficient for the entire family.

In certain embodiments, the comprehensive patient biological information collection device 10 collects patients' biological information through a set of patient biological information collection sensors 400. The set of patient biological information collection sensors 400 constantly monitors the biological information of the patient 30 and transmits the patient biological information collected to the telemedicine system server 21 through the comprehensive patient biological information collection device 10. When at least one of the patient biological information collection sensors 400 detects at least one abnormal condition when at least one type of biological information exceeds a normal range of the set of baseline biological information stored in the patient database 24, the telemedicine system server 21 initiates at least voice communication between the telemedicine system server 21 and the comprehensive patient biological information collection device 10 of the patient 30 through the communication system 23 directly to consult the patient 30.

The patient database 24 is connected to and accessible by the telemedicine system server 21. The patient database 24 stores patient information of a set of patients 30. The patient information includes personal information, medical history, a set of baseline biological information, patient contact information, and contact information of relatives and friends on record of each of the set of the patients 30.

The communication system 23 is connected to the telemedicine system server 21, and provides voice, text, and video communication over a communication network 22 among the patients 30, one or more live telemedicine clinicians 262 from a telemedicine center 26, and one or more patient's relatives and friends on record.

In certain embodiments, the telemedicine system server 21 includes: a server processor 212, a network interface controller 214, and a non-volatile memory 216. The server processor 212 controls operations of the telemedicine system 20. The network interface controller 214 is connected to the communication network 22 through a firewall connector 251 over a firewall 25. The non-volatile memory 216 stores a server operating system 2162, a network communication module 2164, and a telemedicine system controller 2166. The telemedicine system controller 2166 includes a patient information storage module 21662 for accessing the patient database 24 through a database interface 242, a communication control module 21664 for facilitating communication to the communication system 23 through a communication control interface 2301, a patient information processing module 21666, and computer executable instructions 21668. When executed by the server processor 212, the computer executable instructions 21668 perform one or more of following operations:
receiving, through a patient communication module 10661 of the comprehensive patient biological information collection device 10 of the patient 30, patient biological information constantly monitored and collected in real time by the set of patient biological information collection sensors 400 of the comprehensive patient biological information collection device 10;
processing, by the patient information processing module 21666, the patient biological information received;
comparing, by the patient information processing module 21666, the patient biological information processed against base line patient biological information from the patient database 24 using an artificial intelligence algorithm;
initiating, by the telemedicine system server 21, a voice call to the comprehensive patient biological information collection device 10 of the patient 30 through the communication system 23, when at least one of the patient biological information collection sensors 400 detects at least one abnormal condition;
initiating, by the telemedicine system server 21, a call to the telemedicine center 26 to notify the live telemedicine clinician 262 with the patient's GPS location information and patient information including contact information of patient's relatives and friends on record;
contacting, through the communication control module 21664, the patient 30 via the comprehensive patient biological information collection device 10 carried by the patient 30 to perform a remote diagnose and to use an artificial intelligence algorithm to decide whether an immediate or a future telemedicine session with healthcare professional is necessary using the patient information received and retrieved from the patient database 24, the patient biological information collected;
starting, via the comprehensive patient biological information collection device 10, the immediate telemedicine session with the patient 30 by the healthcare professionals, if the immediate telemedicine session is necessary; and
setting up, via the comprehensive patient biological information collection device 10, the future telemedicine session if the immediate telemedicine session is not necessary or required healthcare professionals for the patient 30 is not immediately available.

In certain embodiments, as shown in FIG. 1, each of the set of comprehensive patient biological information collection devices 10 includes: a processor 102, a network interface controller 104, and a non-volatile memory 106. The processor 102 controls operations of the comprehensive patient biological information collection device 10. The network interface controller 104 facilitates the communication among the comprehensive patient biological information collection device 10, the telemedicine system server 21 and the communication system 23 over the communication network 22. The non-volatile memory 106 stores an operating system 1062, a GPS module 1064 for detecting the GPS location of the patient 30 carrying the comprehensive patient biological information collection device 10, and a comprehensive patient biological information collection device controller 1066. The comprehensive patient biological information collection device controller 1066 includes the patient communication module 10661, a comprehensive patient biological information collection module 10663, a comprehensive patient biological information processing module 10665, a comprehensive patient biological information storage module 10667; and computer executable instructions 10669. When executed by the processor 102, the computer executable instructions 10669 perform one or more of following operations:
receiving, through the comprehensive patient biological information collection module 10663 over the patient communication module 10661, patient biological information constantly monitored and collected in real time by the set of patient biological information collection sensors 400;
processing, by the comprehensive patient biological information processing module 10665, the patient biological information received from the set of patient biological information collection sensors 400;
storing, by the comprehensive patient biological information storage module 10667, the processed patient biological information;
and updating, by the patient communication module 10661, the patient database 24.

In certain embodiments, the patient biological information includes: speech samples, facial images and video, electroencephalography (EEG) data, electrocardiography (ECG/EKG) data, breath samples, blood glucose content, blood oxygen content, pulse rate, blood pressure, sound samples such as breathing, heart, thoracic, arterial, intravenous, uterine, fetal, intestinal and other sounds, and body temperatures at various time. The baseline biological information stored in the patient database 24 may include: normal speech samples including tempo, pitch, and intensity of speech, normal facial images and normal walking videos of the patient 30 from many different angles and directions, normal electroencephalography (EEG) data, normal electrocardiography (ECG/EKG) data, normal breath samples, normal blood glucose content range, normal blood oxygen content range, normal pulse rate range, normal blood pressure range, normal sound samples such as breathing, heart, thoracic, arterial, intravenous, uterine, fetal, intestinal and other sounds, and normal body temperatures range at various time.

In certain embodiments, the set of patient biological information collection sensors 400 includes one or more of:
a chip reader 401 for reading a patient information card 301 where patient information is stored;
a geolocation sensor 402 for tracking the geolocation of the patient 30;
a speaker 403 for the patient 30 to make and receive voice calls, and to provide voice instructions to the patient 30;
a stethoscope sensor 404 for monitoring patient's heart, lung and breathing conditions;
a blood oxygen sensor 405 for monitoring patient's blood oxygen level;
a motion sensor 406 for monitoring patient's movement;
an acoustic sensor 407 for the patient 30 to make and receive voice calls, to generate voice samples of the patient 30, and to detect background sounds surrounding the patient 30;
a temperature sensor 408 for monitoring patient's temperature;
a blood pressure sensor 409 for monitoring patient's blood pressure;
a blood sugar sensor 410 for monitoring patient's blood sugar level;
an EKG/ECG sensor 411 for monitoring patient's electrocardiograms;
an EEG sensor 412 for monitoring patient's electroencephalograms;
a video camera 413 for monitoring patient's posture and movement;
an accelerometer 414 for detecting impact to the patient 30 and sudden fall of the patient 30;
an abnormal odor sensor 415 for detecting harmful gas around the patient 30;
a breathalyzer sensor 416 for detecting blood alcohol level of the patient 30, and any combinations of such biological information collection sensors 400.

In certain embodiments, the comprehensive patient biological information collection device 10 also includes: a telemedicine contact button 101, the patient condition indicator 103, a display screen 105, a power supply module 107, and a SIM card slot 109. When an immediate telemedicine session is requested by the patient 30, the patient 30 presses the telemedicine contact button 101 to initiate a contact with the live telemedicine clinician 262. The patient condition indicator 103 is lit in green indicating that the patient 30 is in normal condition. When at least one abnormal condition is detected on the patient 30 and the patient 30 may need healthcare professional's attention, the patient condition indicator 103 turns in red. The display screen 105 is used for receiving and displaying text messages, emails, diagnostic messages, and making video calls. The power supply module 107 provides electrical power to the comprehensive patient biological information collection device 10. A SIM card for mobile communication is inserted in the SIM card slot 109 to enable wireless communication.

In certain embodiments, a patient condition indicator 103 of the comprehensive patient biological information collection device 10 is lit in green light indicating the comprehensive patient biological information collection device 10 is in normal operation state, and the patient 30 is in normal condition. When at least one abnormal condition is detected from the patient 30, the patient condition indicator 103 turns red indicating that the patient 30 should consult at least certain healthcare professionals. At this time, the comprehensive patient biological information collection device 10 of the patient 30 initiates a call to the telemedicine center 26 to notify a live telemedicine clinician 262 with the patient's GPS location information and patient information including contact information of patient's relatives and friends on record, the live telemedicine clinician 262 connects to the patient 30 based on the patient information received and retrieved from the patient database 24. The live telemedicine clinician 262 uses the patient biological information collected to perform a remote diagnose and to use an artificial intelligence algorithm to decide whether an immediate or a future telemedicine session with healthcare professional is necessary using the patient information received and retrieved from the patient database 24, the patient biological information collected.

In certain embodiments, the communication network 22 includes a wireless personal area network (WPAN) and the wireless personal area network includes a Wi-Fi network, a Bluetooth network, an infrared network, and a Zigbee network, a wireless local area network (WLAN), a wireless metropolitan area network (WMAN), a wireless wide area network (WWAN), a cellular network, and a mobile communication network.

In certain embodiments, as shown in FIG. 4, the comprehensive patient biological information collection device 10 includes the SIM card holder 109 to hold a SIM card for mobile communication capable comprehensive patient biological information collection device 10.

In certain embodiments, the communication among the patient 30, the live telemedicine clinician 262, the patient's relatives and friends on record and nearby medical facilities includes: mobile voice calls, mobile videotelephony calls, landline voice calls, videotelephony calls over the Internet, text messages over a mobile phone, text messages over a set of social media platforms, and videotelephony calls over the set of social media platforms.

In certain embodiments, the communication among the patient 30, the live telemedicine clinician 262, the patient's relatives and friends on record and the nearby medical facility includes: mobile voice calls, mobile videotelephony calls, landline voice calls, videotelephony call over the Internet, text messages over mobile phones, text messages over a set of social media platforms, and videotelephony calls over the set of social media platforms.

In certain embodiments, the communication system 23 includes:
a voice input module 231 having a mobile voice input interface 2311 to receive mobile voice calls, and a landline voice input interface 2312 to receive landline voice calls;
a text input module 232 having a social media text input interface 2321 to receive text messages through the set of social media platforms, and a text message input interface 2322 to receive text messages through mobile phones;
a video input module 233 having a mobile video input interface 2331 to receive video calls over the mobile phones, and a social media video input interface 2332 to receive video calls through the set of social media platforms;
a voice output module 234 having a mobile voice output interface 2341 to make mobile voice calls, and a landline voice output interface 2342 to make landline voice calls;
a text output module 235 having a social media text output interface 2351 to transmit text messages through the set of social media platforms, and a text message output interface 2352 to transmit text messages through the mobile phones;
a video output module 236 having a mobile video output interface 2361 to make video calls over the mobile phones, and a social media video output interface 2362 to make video calls through the set of social media platforms;
a speech to text conversion module 237 for converting voice input to text input;
a live telemedicine clinician interface module 238 for the live telemedicine clinician 262 to receive and make conference calls among the patient 30, the live telemedicine clinician 262, the patient's relatives and friends on record and the nearby medical facility through voice calls, text messages, and video calls; and
a text to speech conversion module 239 to make voice calls to the patient 30 through the comprehensive patient biological information collection device 10.

In certain embodiments, the social media platforms include, but not limited to: Facebook, YouTube, Telegram, Parler, WhatsApp, Messenger, WeChat, Instagram, QQ, Tumblr, Qzone, Tik Tok, Sina Weibo, Twitter, Reddit, Baidu Tieba, LinkedIn, Viber, Snapchat, and Pinterest and various combination of these social media platforms.

In certain embodiments, the communication control interface 2301 includes: a voice input terminal 23011 connected to the voice input module 231, a text input terminal 23012 connected to the text input module 232, a video input terminal 23013 connected to the video input module 233, a voice output terminal 23014 connected to the voice output module 234, a text output terminal 23015 connected to the text output module 235, and a video output terminal 23016 connected to the video output module 236.

In certain embodiments, the comprehensive patient biological information collection device 10 includes:
a portable comprehensive patient biological information collection device to be carried with the patient 30;
a stationary comprehensive patient biological information collection device to be placed at home or work place of the patient 30 and to be shared with one or more registered patients 30; and
a set of public stationary comprehensive patient biological information collection devices to be placed in public places and to be shared with one or more registered patients 30.

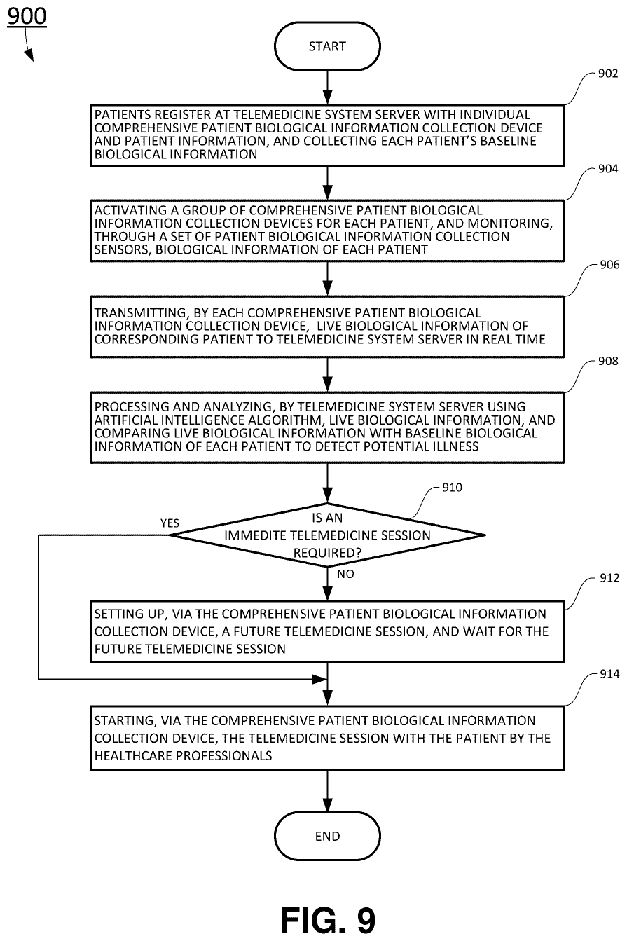

In yet another aspect, as shown in FIG. 9, the present disclosure relates to a method of using a telemedicine system 20 having a set of comprehensive patient biological information collection devices 10. In certain embodiments, the method includes:
registering, by a set of patients 30, the set of patients 30, and a set of comprehensive patient biological information collection devices 10 at a telemedicine system server 21 of the telemedicine system 20, wherein patient information of each of the set of patients 30 is stored in a patient database 24 of the telemedicine system 20, and the telemedicine system 20 provides telemedicine assistance to the patient 30 by a healthcare professional when a comprehensive patient biological information collection device 10 of the patient 30 detects at least one abnormal condition when at least one type of biological information exceeds a normal range of the set of baseline biological information stored in the patient database 24;
constantly monitoring and collecting in real time, by a set of patient biological information collection sensors 400 of the set of the comprehensive patient biological information collection devices 10, patient biological information of each patient 30, where the patient 30 logs into his/her own registered account;
receiving, through a patient communication module 10661 from the comprehensive patient biological information collection device 10, the patient biological information constantly monitored and collected in real time by the set of patient biological information collection sensors 400 of the comprehensive patient biological information collection device 10;
processing, through a patient information processing module 21666 of the telemedicine system server 21 of the telemedicine system 20, the patient biological information received, and comparing the patient biological information received with a set of baseline biological information stored in the patient database 24 using an artificial intelligence algorithm;
initiating, by the telemedicine system server 21, a voice call to the comprehensive patient biological information collection device 10 of the patient 30 through a communication system 23, when at least one of the patient biological information collection sensors 400 detects at least one abnormal condition;
initiating, by the telemedicine system server 21, a call to the telemedicine center 26 to notify the live telemedicine clinician 262 with the patient's GPS location information and patient information including contact information of patient's relatives and friends on record;
contacting, through a communication control module 21664 of a telemedicine system controller 2166 of the telemedicine system server 21, the patient 30 via the comprehensive patient biological information collection device 10 carried by the patient 30 to perform a remote diagnose and to use an artificial intelligence algorithm to decide whether an immediate or a future telemedicine session with healthcare professional is necessary using the patient information received and retrieved from the patient database 24, the patient biological information collected;
starting, via the comprehensive patient biological information collection device 10, the immediate telemedicine session with the patient 30 by the healthcare professionals, if the immediate telemedicine session is necessary; and
setting up, via the comprehensive patient biological information collection device 10, the future telemedicine session if the immediate telemedicine session is not necessary or required healthcare professionals for the patient 30 is not immediately available.

In certain embodiments, the starting, via the comprehensive patient biological information collection device 10, the immediate telemedicine session with the patient 30 by the healthcare professionals includes:
establishing voice communication, text messages or video communication, via the communication system 23 of the telemedicine system 20, between a healthcare professional and the patient 30;
connecting, via the communication system 23 of the telemedicine system 20, the plurality of patient biological information collection sensors 400 through the comprehensive patient biological information collection device 10; and
performing, by the healthcare professional via the comprehensive patient biological information collection device 10, a diagnose of the patient 30 using the patient's biological information retrieved from the patient database 24, the patient's biological information collected through the plurality of patient biological information collection sensors 400, and the feedback from the patient 30; and
providing timely treatment, by the healthcare professional via the comprehensive patient biological information collection device 10, to the patient 30.

FIG. 9 shows a flow chart of the method 900 of using the telemedicine system 20 having a set of comprehensive patient biological information collection devices 10 according to certain embodiments of the present disclosure.

At block 902, each of a group of patients 30 registers a personal account with at a telemedicine system server 21 of the telemedicine system 20. Patient's patient information is entered and stored in a patient database 24 during the registeration. The patient information includes personal information, medical history, a set of baseline biological information, patient contact information, and contact information of relatives and friends on record of each of the set of the patients.

The comprehensive patient biological information collection device 10 may be used by one patient 30 at a time. Each comprehensive patient biological information collection device 10 has to be registered at the telemedicine system server 21 of the telemedicine system 20 as well. When the comprehensive patient biological information collection device 10 is in use, the patient 30 has to log into his/her own account with the comprehensive patient biological information collection device 10.

At block 904, each of the comprehensive patient biological information collection device 10 is activated at the telemedicine system server 21 of the telemedicine system 20, and each of the comprehensive patient biological information collection device 10 constantly monitors and collects in real time biological information of each patient 30 through a set of patient biological information collection sensors 400 of the comprehensive patient biological information collection device 10.

In certain embodiments, the patient biological information includes: speech samples, facial images and video, electroencephalography (EEG) data, electrocardiography (ECG/EKG) data, breath samples, blood glucose content, blood oxygen content, pulse rate, blood pressure, sound samples such as breathing, heart, thoracic, arterial, intravenous, uterine, fetal, intestinal and other sounds, and body temperatures at various time.

At block 906, live biological information of each patient 30 collected by the set of patient biological information collection sensors 400 is transmitted to the telemedicine system server 21 of the telemedicine system 20 through a communication network 22 in real time.

At block 908, the telemedicine system server 21 processes and analyzes the live patient biological information received from each patient 30 using artificial intelligence algorithms and comparing the live patient biological information received from each patient 30 with corresponding patient baseline biological information stored in the patient database 24.

At query block 910, based on the live patient biological information collected, patient medical history, and other patient information, the telemedicine system server 21 uses an artificial intelligence algorithm to decide whether an immediate or a future telemedicine session with healthcare professional is necessary using the patient information received and retrieved from the patient database 24, the patient biological information collected.

At block 912, when the immediate telemedicine session is not necessary or required healthcare professional for the patient 30 is not immediately available, a future telemedicine session is set up by the telemedicine system server 21 via the comprehensive patient biological information collection device 10.

At block 914, when the immediate telemedicine session is necessary, or when telemedicine session time is up, a healthcare professional starts the telemedicine session with the patient 30 using the comprehensive patient biological information collection device 10.

In certain embodiments, the telemedicine session includes:
establishing voice communication, text messages or video communication, via the communication system 23 of the telemedicine system 20, between the healthcare professional and the patient 30;
connecting, via the communication system 23 of the telemedicine system 20, the set of patient biological information collection sensors 400 through the comprehensive patient biological information collection device 10; and
performing, by the healthcare professional via the comprehensive patient biological information collection device 10, a diagnose of the patient 30 using the patient's biological information retrieved from the patient database 24, the patient's biological information collected through the set of patient biological information collection sensors 400, and the feedback from the patient 30; and
providing timely treatment, by the healthcare professional via the comprehensive patient biological information collection device 10, to the patient 30.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope. Accordingly, the scope of the present disclosure is defined by the appended claims rather than the foregoing description and the exemplary embodiments described therein.

What is claimed is:

1. A comprehensive patient biological information collection device comprises:
a processor, wherein the processor controls operations of the comprehensive patient biological information collection device;
a network interface controller, wherein the network interface controller facilitates the communication among the comprehensive patient biological information collection device, a telemedicine system server and a communication system over a communication network; and
a non-volatile memory, wherein the non-volatile memory stores an operating system, a GPS module for detecting the GPS location of a patient carrying the comprehensive patient biological information collection device, and a comprehensive patient biological information collection device controller having a patient communication module, a comprehensive patient biological information collection module, a comprehensive patient biological information processing module, a comprehensive patient biological information storage module; and computer executable instructions, when executed by the processor, the computer executable instructions perform one or more of following operations:
receiving, through the comprehensive patient biological information collection module over the patient communication module, patient biological information constantly monitored and collected in real time by a plurality of patient biological information collection sensors of the comprehensive patient biological information collection device;
processing, by the comprehensive patient biological information processing module, the patient biological information received from the plurality of patient biological information collection sensors;
storing, by the comprehensive patient biological information storage module, the processed patient biological information; and
updating, through the patient communication module, a patient database.

2. The comprehensive patient biological information collection device according to claim 1, wherein
the patient communication module facilitates communication among the patient database, the comprehensive patient biological information collection device controller, and the plurality of patient biological information collection sensors through the network interface controller over the communication network;
the comprehensive patient biological information collection module collects patient biological information through a wireless data receiver over the communication network from the plurality of patient biological information collection sensors from the patient;
the comprehensive patient biological information processing module processes the patient biological information collected through the comprehensive patient biological information collection module; and
the comprehensive patient biological information storage module stores the patient biological information processed through the comprehensive patient biological information processing module and synchronizes the patient biological information stored with the patient database.

3. The comprehensive patient biological information collection device according to claim 1, wherein the communication network comprises a wireless personal area network (WPAN) having a Wi-Fi network, a Bluetooth network, an infrared network, and a Zigbee network, a wireless local area network (WLAN), a wireless metropolitan area network (WMAN), a wireless wide area network (WWAN), a cellular network, and a mobile communication network.

4. The comprehensive patient biological information collection device according to claim 3, wherein the plurality of patient biological information collection sensors comprises one or more of:
a chip reader, wherein the chip reader reads a patient information card where patient information is stored;
a geolocation sensor, wherein the geolocation sensor tracks the geolocation of the patient;
a speaker, wherein the speaker is used for the patient to make and receive voice calls, and to provide voice instructions to the patient;
a stethoscope sensor, wherein the stethoscope sensor monitors patient's heart, lung and breathing conditions;
a blood oxygen sensor, wherein the blood oxygen sensor monitors patient's blood oxygen level;
a motion sensor, wherein the motion sensor monitors patient's movement;
an acoustic sensor, wherein the acoustic sensor is used by the patient to make and receive voice calls, to generate voice samples of the patient, and to detect background sounds surrounding the patient;
a temperature sensor, wherein the temperature sensor monitors patient's temperature;
a blood pressure sensor, wherein the blood pressure sensor monitors patient's blood pressure;
a blood sugar sensor, wherein the blood sugar sensor monitors patient's blood sugar level;
an EKG/ECG sensor, wherein the EKG/ECG sensor monitors patient's electrocardiograms;
an EEG sensor, wherein the EEG sensor monitors patient's electroencephalograms;
a video camera, wherein the video camera monitors patient's posture and movement;
an accelerometer, wherein the accelerometer detects impact to the patient and sudden fall of the patient;
an abnormal odor sensor, wherein the abnormal odor sensor detects harmful gas around the patient;
a breathalyzer sensor, wherein the breathalyzer sensor detects blood alcohol level of the patient; and
any combinations thereof.

5. A telemedicine system, comprising:
a telemedicine system server, wherein each of a plurality of patients and each of a plurality of comprehensive patient biological information collection device are registered at the telemedicine system server, wherein one comprehensive patient biological information collection device is used by one or more patients, and only one patient who has logged into his/her own registered account can use the comprehensive patient biological information collection device, the telemedicine system provides telemedicine assistance to a patient by a healthcare professional when the comprehensive patient biological information collection device of the patient detects at least one abnormal condition from the patient;
a patient database, wherein the patient database is connected to and accessible by the telemedicine system server, wherein the patient database stores patient information of the plurality of patients, wherein the patient information comprises personal information, medical history, a set of baseline biological information, patient contact information, and contact information of relatives and friends on record of each of the plurality of the patients;
a communication system, wherein the communication system is connected to the telemedicine system server, wherein the communication system provides voice, text, and video communication over a communication network among the patient, one or more live telemedicine clinicians from a telemedicine center, and one or more patient's relatives and friends on record;
a plurality of comprehensive patient biological information collection devices, wherein each comprehensive patient biological information collection device is logged into his/her own registered account and used by one patient at a time, and the comprehensive patient biological information collection device collects patients' biological information through a plurality of patient biological information collection sensors, the plurality of patient biological information collection sensors constantly monitors the biological information of the patient and transmits the patient biological information collected to the telemedicine system server through the comprehensive patient biological information collection device, and when at least one of the patient biological information collection sensors detects an abnormal condition when at least one type of biological information exceeds a normal range of the set of baseline biological information stored in the patient database, the telemedicine system server initiates at least voice communication between the telemedicine system server and the comprehensive patient biological information collection device of the patient through the communication system directly to consult the patient,
wherein a patient condition indicator of the comprehensive patient biological information collection device is lit in green light indicating the comprehensive patient biological information collection device is in normal operation state, and the patient is in normal condition, when at least one abnormal condition is detected from the patient, the patient condition indicator turns red indicating that the patient should consult at least certain healthcare professionals, the comprehensive patient biological information collection device initiates a call to the telemedicine center to notify a live telemedicine clinician with the patient's GPS location information and patient information including contact information of patient's relatives and friends on record, the live telemedicine clinician connects to the patient based on the patient information received and retrieved from the patient database, and the live telemedicine clinician uses the patient biological information collected to perform a remote diagnose and to use an artificial intelligence algorithm to decide whether an immediate or a future telemedicine session with healthcare professional is necessary using the patient information received and retrieved from the patient database, the patient biological information collected.

6. The telemedicine system according to claim 5, wherein the communication network comprises a wireless personal area network (WPAN) having a Wi-Fi network, a Bluetooth network, an infrared network, and a Zigbee network, a wireless local area network (WLAN), a wireless metropolitan area network (WMAN), a wireless wide area network (WWAN), a cellular network, and a mobile communication network.

7. The telemedicine system according to claim 5, wherein the telemedicine system server comprises:
a server processor for controlling operations of the telemedicine system;
a network interface controller connected to the communication network through a firewall connector over a firewall; and
a non-volatile memory for storing an server operating system, a network communication module, and a telemedicine system controller having a patient information storage module for accessing the patient database through a database interface, a communication control module for facilitating communication to the communication system through a communication control interface, a patient information processing module, and computer executable instructions, when executed by the server processor, the computer executable instructions perform one or more of following operations:
receiving, through a patient communication module of the comprehensive patient biological information collection device of the patient, patient biological information constantly monitored and collected in real time by the plurality of patient biological information collection sensors of the comprehensive patient biological information collection device;
processing, by the patient information processing module, the patient biological information received;
comparing, by the patient information processing module, the patient biological information processed against base line patient biological information from the patient database using an artificial intelligence algorithm;
initiating, by the telemedicine system server, a voice call to the comprehensive patient biological information collection device of the patient through the communication system, when at least one of the patient biological information collection sensors detects at least one abnormal condition when at least one type of biological information exceeds a normal range of the set of baseline biological information stored in the patient database;
initiating, by the telemedicine system server, a call to the telemedicine center to notify the live telemedicine clinician with the patient's GPS location information and patient information including contact information of patient's relatives and friends on record;
contacting, through the communication control module, the patient via the comprehensive patient biological information collection device carried by the patient to perform a remote diagnose and to use an artificial intelligence algorithm to decide whether an immediate or a future telemedicine session with healthcare professional is necessary using the patient information received and retrieved from the patient database, the patient biological information collected;
starting, via the comprehensive patient biological information collection device, the immediate telemedicine session with the patient by the healthcare professionals, if the immediate telemedicine session is necessary; and
setting up, via the comprehensive patient biological information collection device, the future telemedicine session if the immediate telemedicine session is not necessary or required healthcare professionals for the patient is not immediately available.

8. The telemedicine system according to claim 5, wherein each of the plurality of comprehensive patient biological information collection devices comprises:
a processor, wherein the processor controls operations of the comprehensive patient biological information collection device;
a network interface controller, wherein the network interface controller facilitates the communication among the comprehensive patient biological information collection device, the telemedicine system server and the communication system over the communication network; and
a non-volatile memory, wherein the non-volatile memory stores an operating system, a GPS module for detecting the GPS location of the patient carrying the comprehensive patient biological information collection device, and a comprehensive patient biological information collection device controller having the patient communication module, a comprehensive patient biological information collection module, a comprehensive patient biological information processing module, a comprehensive patient biological information storage module; and computer executable instructions, when executed by the processor, the computer executable instructions perform one or more of following operations:
receiving, through the comprehensive patient biological information collection module over the patient communication module, patient biological information constantly monitored and collected in real time by the plurality of patient biological information collection sensors;
processing, by the comprehensive patient biological information processing module, the patient biological information received from the plurality of patient biological information collection sensors;
storing, by the comprehensive patient biological information storage module, the processed patient biological information;
and updating, by the patient communication module, the patient database.

9. The telemedicine system according to claim 8, wherein the plurality of patient biological information collection sensors comprises one or more of:
a chip reader, wherein the chip reader reads a patient information card where patient information is stored;
a geolocation sensor, wherein the geolocation sensor monitors the geolocation of the patient;
a speaker, wherein the speaker is used for the patient to make and receive voice calls, and to provide voice instructions to the patient;
a stethoscope sensor, wherein the stethoscope sensor monitors patient's heart, lung and breathing conditions;
a blood oxygen sensor, wherein the blood oxygen sensor monitors patient's blood oxygen level;
a motion sensor, wherein the motion sensor monitors patient's movement;
an acoustic sensor, wherein the acoustic sensor is used by the patient to make and receive voice calls, to generate voice samples of the patient, and to detect background sounds surrounding the patient;
a temperature sensor, wherein the temperature sensor monitors patient's temperature;
a blood pressure sensor, wherein the blood pressure sensor monitors patient's blood pressure;
a blood sugar sensor, wherein the blood sugar sensor monitors patient's blood sugar level;
an EKG/ECG sensor, wherein the EKG/ECG sensor monitors patient's electrocardiograms;
an EEG sensor, wherein the EEG sensor monitors patient's electroencephalograms;
a video camera, wherein the video camera monitors patient's posture and movement;
an accelerometer, wherein the accelerometer detects impact to the patient and sudden fall of the patient;
an abnormal odor sensor, wherein the abnormal odor sensor detects harmful gas around the patient;
a breathalyzer sensor, wherein the breathalyzer sensor detects blood alcohol level of the patient; and
any combinations thereof.

10. The telemedicine system according to claim 5, wherein the comprehensive patient biological information collection device further comprises:
a telemedicine contact button, wherein the patient presses the telemedicine contact button to initiate a contact with the live telemedicine clinician when an immediate telemedicine session is requested by the patient;
the patient condition indicator; wherein the patient condition indicator is lit in green indicating that the patient is in normal condition, and the patient condition indicator turns in red indicating that at least one abnormal condition is detected on the patient and the patient may need healthcare professional's attention;
a display screen, wherein the display screen receives and displays text messages, emails, diagnostic messages, and carries out video calls;
a power supply module, wherein the power supply module provides electrical power to the comprehensive patient biological information collection device; and
a SIM card slot, wherein a SIM card for mobile communication is inserted in the SIM card slot to enable wireless communication.

11. The telemedicine system according to claim 5, wherein the communication among the patient, the live telemedicine clinician, the patient's relatives and friends on record and nearby medical facilities comprises:
mobile voice calls;
mobile videotelephony calls;
landline voice calls;
videotelephony calls over the Internet;
text messages over a mobile phone;
text messages over a plurality of social media platforms; and
videotelephony calls over the plurality of social media platforms.

12. The telemedicine system according to claim 11, wherein the communication system comprises:
a voice input module having a mobile voice input interface to receive mobile voice calls, and a landline voice input interface to receive landline voice calls;
a text input module having a social media text input interface to receive text messages through the plurality of social media platforms, and a text message input interface to receive text messages through mobile phones;
a video input module having a mobile video input interface to receive video calls over the mobile phones, and a social media video input interface to receive video calls through the plurality of social media platforms;
a voice output module having a mobile voice output interface to make mobile voice calls, and a landline voice output interface to make landline voice calls;
a text output module having a social media text output interface to transmit text messages through the plurality of social media platforms, and a text message output interface to transmit text messages through the mobile phones;
a video output module having a mobile video output interface to make video calls over the mobile phones, and a social media video output interface to make video calls through the plurality of social media platforms;
a speech to text conversion module for converting voice input to text input;
a live telemedicine clinician interface module for the live telemedicine clinician to receive and make conference calls among the patient, the live telemedicine clinician, the patient's relatives and friends on record and nearby medical facilities through voice calls, text messages, and video calls; and
a text to speech conversion module to make voice calls to the patient through the comprehensive patient biological information collection device.

13. The telemedicine system according to claim 12, wherein the communication control interface comprises:
a voice input terminal connected to the voice input module;
a text input terminal connected to the text input module;
a video input terminal connected to the video input module;
a voice output terminal connected to the voice output module;
a text output terminal connected to the text output module; and
a video output terminal connected to the video output module.

14. The telemedicine system according to claim 5, wherein the comprehensive patient biological information collection device comprises:
a portable comprehensive patient biological information collection device to be carried with the patient;
a stationary comprehensive patient biological information collection device to be placed at home or work place of the patient and to be shared with one or more registered patients; and
a plurality of public stationary comprehensive patient biological information collection devices to be placed in public places and to be shared with one or more registered patients.

15. A method of using a telemedicine system, comprising:
registering, by a plurality of patients, the plurality of patients and a plurality of comprehensive patient biological information collection devices at a telemedicine system server of the telemedicine system, wherein patient information of each of the plurality of patients is stored in a patient database of the telemedicine system, and the telemedicine system provides telemedicine assistance to the patient by a healthcare professional when a comprehensive patient biological information collection device of the patient detects at least one abnormal condition when at least one type of biological information exceeds a normal range of the set of baseline biological information stored in the patient database;
constantly monitoring and collecting in real time, by a plurality of patient biological information collection sensors of the plurality of the comprehensive patient biological information collection devices, patient biological information of each patient, one patient at a time, where the patient logs into his/her own registered account;
receiving, through a patient communication module from the comprehensive patient biological information collection device, the patient biological information constantly monitored and collected in real time by the plurality of patient biological information collection sensors of the comprehensive patient biological information collection device;
processing, through a patient information processing module of the telemedicine system server of the telemedicine system, the patient biological information received, and comparing the patient biological information received with a set of baseline biological information stored in the patient database using an artificial intelligence algorithm;
initiating, by the telemedicine system server, a voice call to the comprehensive patient biological information collection device of the patient through a communication system, when at least one of the patient biological information collection sensors detects at least one abnormal condition;
initiating, by the telemedicine system server, a call to a telemedicine center to notify the live telemedicine clinician with the patient's GPS location information and patient information including contact information of patient's relatives and friends on record;
contacting, through a communication control module of a telemedicine system controller of the telemedicine system server, the patient via the comprehensive patient biological information collection device carried by the patient to perform a remote diagnose and to use an artificial intelligence algorithm to decide whether an immediate or a future telemedicine session with healthcare professional is necessary using the patient information received and retrieved from the patient database, the patient biological information collected;
starting, via the comprehensive patient biological information collection device, the immediate telemedicine session with the patient by the healthcare professionals, if the immediate telemedicine session is necessary; and
setting up, via the comprehensive patient biological information collection device, the future telemedicine session if the immediate telemedicine session is not necessary or required healthcare professionals for the patient is not immediately available.

16. The method of claim 15, wherein the starting, via the comprehensive patient biological information collection device, the immediate telemedicine session with the patient by the healthcare professionals comprises:
establishing voice communication, text messages or video communication, via the communication system of the telemedicine system, between a healthcare professional and the patient;
connecting, via the communication system of the telemedicine system, the plurality of patient biological information collection sensors through the comprehensive patient biological information collection device; and
performing, by the healthcare professional via the comprehensive patient biological information collection device, a diagnose of the patient using the patient's biological information retrieved from the patient database, the patient's biological information collected through the plurality of patient biological information collection sensors, and the feedback from the patient; and
providing timely treatment, by the healthcare professional via the comprehensive patient biological information collection device, to the patient.

17. The method of claim 15, wherein the telemedicine system comprises:
the telemedicine system server, wherein each of the plurality of patients and each of a plurality of comprehensive patient biological information collection device are registered at the telemedicine system server wherein a comprehensive patient biological information collection device is shared by one or more patients, and the comprehensive patient biological information collection device is used by only one logged in patient at a time, the telemedicine system provides telemedicine assistance to a patient by a healthcare professional when the comprehensive patient biological information collection device of the patient detects at least one abnormal condition from the patient;
the patient database, wherein the patient database is connected to and accessible by the telemedicine system server, wherein the patient database stores patient information of the plurality of patients, wherein the patient information comprises personal information, medical history, patient contact information, and contact information of relatives and friends on record of each of the plurality of the patients;
the communication system, wherein the communication system is connected to the telemedicine system server, wherein the communication system provides voice, text, and video communication over a communication network among the patient, one or more live telemedicine clinicians from the telemedicine center, and one or more patient's relatives and friends on record; and
the plurality of comprehensive patient biological information collection devices, wherein each of the plurality of patients carries one corresponding comprehensive patient biological information collection device, and each comprehensive patient biological information collection device collects patients' biological information through the plurality of patient biological information collection sensors and the set of patient biological information collection sensors constantly monitors the biological information of the patient and transmits the patient biological information collected to the telemedicine system server through the comprehensive patient biological information collection device, when at least one of the patient biological information collection sensors detects at least one abnormal condition when at least one type of biological information exceeds a normal range of the set of baseline biological information stored in the patient database, the telemedicine system server initiates at least voice communication between the telemedicine system server and the comprehensive patient biological information collection device of the patient through the communication system directly.

18. The method of claim 17, wherein the comprehensive patient biological information collection device comprises:
a processor, wherein the processor controls operations of the comprehensive patient biological information collection device;
a network interface controller, wherein the network interface controller facilitates the communication among the comprehensive patient biological information collection device, the telemedicine system server and the communication system over the communication network; and
a non-volatile memory, wherein the non-volatile memory stores an operating system, a GPS module for detecting the GPS location of the patient carrying the comprehensive patient biological information collection device, and a comprehensive patient biological information collection device controller having a patient communication module, a comprehensive patient biological information collection module, a comprehensive patient biological information processing module, a comprehensive patient biological information storage module; and computer executable instructions, when executed by the processor, the computer executable instructions perform one or more of following operations:
receiving, through the comprehensive patient biological information collection module over the patient communication module, patient biological information constantly monitored and collected in real time by the plurality of patient biological information collection sensors of the comprehensive patient biological information collection device;
processing, by the comprehensive patient biological information processing module, the patient biological information received from the plurality of patient biological information collection sensors;
storing, by the comprehensive patient biological information storage module, the processed patient biological information;
and updating, by the patient communication module, the patient database of the telemedicine system.

19. The method of claim 18, wherein the plurality of patient biological information collection sensors comprises one or more of:
a chip reader, wherein the chip reader reads a patient information card where patient information is stored;
a geolocation sensor, wherein the geolocation sensor monitors the geolocation of the patient;
a speaker, wherein the speaker is used for the patient to make and receive voice calls, and to provide voice instructions to the patient;
a stethoscope sensor, wherein the stethoscope sensor monitors patient's heart, lung and breathing conditions;
a blood oxygen sensor, wherein the blood oxygen sensor monitors patient's blood oxygen level;
a motion sensor, wherein the motion sensor monitors patient's movement;
an acoustic sensor, wherein the acoustic sensor is used by the patient to make and receive voice calls, to generate voice samples of the patient, and to detect background sounds surrounding the patient;
a temperature sensor, wherein the temperature sensor monitors patient's temperature;
a blood pressure sensor, wherein the blood pressure sensor monitors patient's blood pressure;
a blood sugar sensor, wherein the blood sugar sensor monitors patient's blood sugar level;
an EKG/ECG sensor, wherein the EKG/ECG sensor monitors patient's electrocardiograms;
an EEG sensor, wherein the EEG sensor monitors patient's electroencephalograms;
a video camera, wherein the video camera monitors patient's posture and movement;
an accelerometer, wherein the accelerometer detects impact to the patient and sudden fall of the patient;
an abnormal odor sensor, wherein the abnormal odor sensor detects harmful gas around the patient;
a breathalyzer sensor, wherein the breathalyzer sensor detects blood alcohol level of the patient; and
any combinations thereof.

20. The method of claim 19, wherein the comprehensive patient biological information collection device comprises:
a portable comprehensive patient biological information collection device to be carried with the patient;
a stationary comprehensive patient biological information collection device to be placed at home or work place of the patient and to be shared with one or more registered patients; and
a set of public stationary comprehensive patient biological information collection devices to be placed in public places and to be shared with one or more registered patients.
